(19) United States
(12) Patent Application Publication
(10) Pub. No.: US 2022/0177834 A1
(43) Pub. Date: Jun. 9, 2022

(54) STABLE SYSTEM FOR THE IN VITRO CULTURE OF CEREBELLAR GRANULE CELL PRECURSORS (GCP), STABLE METHOD FOR THE IN VITRO CULTURE OF SAID CELLS AND USE OF SAID SYSTEM OR METHOD FOR IN VITRO CULTURE

(71) Applicants: UNIVERSITA DEGLI STUDI Dl ROMA "LA SAPIENZA", Rome (IT); FONDAZIONE ISTITUTO ITALIANO Dl TECNOLOGIA, Genova (IT)

(72) Inventors: Giuseppe GIANNINI, Roma (IT); Valeria COLICCHIA, Castiglione Messer Raimondo (IT); Carlo CAPALBO, Roma (IT); Francesca FABRETTI, Formello (IT); Stefano DI GIULIO, Roma (IT); Francesca BELARDINILLI, Ceccano (IT); MariaLaura PETRONI, Roma (IT); Maria Sahùn RONCERO, Zaragoza (ES)

(21) Appl. No.: 17/598,159

(22) PCT Filed: Mar. 19, 2020
(86) PCT No.: PCT/IB2020/052513

§ 371 Date: Sep. 24, 2021

(30) Foreign Application Priority Data
Mar. 26, 2019 (IT) 102019000004377

(51) Int. Cl. C12N 5/0793

(52) U.S. Cl. CPC C12N 5/0619; C12N 2506/08; C12N 2501/11

(57) ABSTRACT

This invention relates to an in vitro system for the culture of cerebellar granule cell precursors (granule cell progenitors, GCP) comprising a culture support, mammalian GCP cells and a culture medium comprising at least SAG (Smoothened agonist) and EGF (Epidermal Growth Factor), and a method for the in vitro culture of GCP cells, use of the above-mentioned culture system or method of culture for the generation of in vitro models for study of the pathophysiology of cerebellar granules or use of the above-mentioned culture system or method of culture for use in gene therapy and cell therapy approaches to cerebellar diseases caused by damage or neurodegeneration.

### FIELD OF INVENTION

This invention relates to

an in vitro culture system for cerebellar granule cell precursors (GCP) including:
a culture medium,
mammalian GCP cells and
a culture medium comprising at least SAG (Smoothened agonist) and EGF (Epidermal Growth Factor);

a method for the in vitro culture of GCP cells including the steps of:

(a) isolation of GCP cells from a mammalian cerebellum,
(b) placing the GCP cells obtained in stage (a) in a culture medium containing at least SAG and EGF,
(c) enrichment of GCP cells and/or propagation of GCP cells by dilution; and use of the culture system or culture method for the generation of in vitro models for study of the pathophysiology of cerebellar granules, or use of the culture system or culture method in gene therapy and cell therapy approaches to cerebellar diseases arising from damage or neurodegeneration.

### PRIOR ART

The cerebellum is the organ for maintaining balance, posture and voluntary movements. This organ is delimited by two germinal zones responsible for the formation of its cells: the Ventricular Zone (VZ) and the Rhombic Lip (RL).

Purkinje cells, Golgi cells and the neurons of the deep nuclei are generated from the Ventricular Zone.

The precursor cells of cerebellar granules (GCP) are generated from the Rhombic Lip.

During murine development (around embryonic stage E10) and human development a layer of GCP cells enter a phase of intense proliferation, migrate tangentially along the surface of the cerebellar primordium (stage E15) and form a layer that covers the entire cerebellum, called the External Germinal Layer (or EGL). This layer expands up to the first 10 days of post-natal life, then GCPs begin to differentiate into mature granules, migrate through the Purkinje cell layer and form the Inner Granular Layer (IGL).

In this respect it should be noted that the processes of multiplication, migration and differentiation of GCP cells are essential for proper development and functioning of the cerebellum and defects in the molecular pathways that regulate them cause various diseases (see https://medline.plus.gov/cerebellardisorders.html). In this respect developmental disorders (such as Ataxia Telangiectasia, Joubert's syndrome, Dandy-Walker syndrome), neurodegenerative diseases (such as spinocerebellar ataxia) and neoplastic transformation (such as medulloblastoma) have been reported.

Countless studies are known from the literature on the molecular pathways that drive the proliferation, migration and differentiation of GCP, but these pathways have not yet been fully understood.

This is also due to the lack of appropriate in vitro study models that fully preserve physiological characteristics while still allowing regulatory pathways to be studied.

One of the fundamental pathways that guides the multiplication of GCP cells is the Sonic Hedgehog (Shh) pathway, so much so that absence of its control is associated with the onset of medulloblastoma (paediatric cancer often due to the uninterrupted proliferation of GCP cells), in both humans and in mouse models.

In the known art cellular models of medulloblastoma that depend on the Shh pathway have been used as models for the study of GCP cells and the molecular mechanisms of that pathway, but it should be noted that the normal cell culture conditions used in these models actually suppress the Shh pathway, making said models completely unsuitable for representing not only the pathology from which they originated but also the physiology of GCP cells.

An in, vitro GCP cell study model widely used in the known art is made up of transient cultures of murine GCP cells (taken from mice at P7) caused to aggregate and stimulated with synthetic-Shhh peptides or with chemical path agonists, such as SAG (Smoothened Agonist).

However these cultures spontaneously differentiate into granules that do not proliferate within a few days (typically 5-6), and are therefore not very stable over time.

Work by the present inventors, still unpublished, indicates that GCP cells from mice (taken from mice at P7) can be maintained longer in the form of neurospheres after stimulation with SAG, but even this condition does not ensure a cell culture that is stable in the long term.

Thus, in the short and especially in the long term, the stability of GCP cell cultures is a major problem in this area.

Research workers studying GCP cells or the Shh route must therefore necessarily create new cultures, sacrificing experimental animals, with an impact on experimental variability and research time and cost.

Hitherto GCP cell cultures in serum-free medium enriched with Epidermal Growth Factor (EGF) and Fibroblast Growth Factor (FGF) have been known. These cultures, although stable over time, do not express physiological pathways typical of GCP (such as the Shh pathway) and in differentiation experiments they constitute a mixed cell population with a higher percentage of glial than neuronal component.

It is in fact known that FGF inhibits the Shh pathway and limits the growth of cerebellar granule precursors.

Accordingly the cultures obtained in this way differ significantly from GCP cells in gene/biochemical expression pattern and the system is heavily contaminated with non-neuronal cell populations.

These cultures are therefore unreliable and not very reproducible.

Thus the said in vitro method of culture and the study model are not properly representative of GCP cells, even though commonly used.

Until now, therefore, in the art no system or method is known for the in vitro culture of GCP cells which is at the same time stable, reliable, reproducible, easy to prepare, and one that allows not only the easy enrichment or propagation of GCP cells, but also for them to be maintained for even very long periods over time and for GCP cells with optimal gene and biochemical expression to be obtained.

There is therefore a need for a system and method for the in vitro culture of GCP cells that overcomes all the disadvantages of the known art mentioned above.

### SUMMARY OF THE INVENTION

Surprisingly, a system and method for the in vitro culture of GCP cells that overcomes all the disadvantages of the systems and/or methods for the in vitro culture of GCP cells known in the art has been found.

In particular, the system and method for the in vitro culture of GCP cells according to the present invention are simple, reliable, easily reproducible and extremely stable (over time, but also with respect to the gene/biochemical expression profile of the GCP cell cultures obtained, very similar to that of fresh GCP cell cultures, and also with respect to the possibility of freezing and thawing them, preservation methods that do not alter their characteristics).

Thus a first object of the present invention is an in vitro system for the culture of cerebellar granule cell precursors (GCP) including:
a culture medium,
mammalian GCP cells and
a culture medium comprising at least SAG (Smoothened agonist) and EGF (Epidermal Growth Factor).

A second object of the present invention is a method for the in vitro culture of GCP cells including the steps of:

(a) isolation of GCP cells from the cerebellum of a mammal,
(b) placing the individual GCP cells obtained in step (a) on a culture medium containing at least SAG and EGF,
(c) enrichment and/or propagation of GCP cells by dilution.

Another object of this invention is use of the culture system or method of in vitro culture for the generation of in vitro models for study of the pathophysiology of cerebellar granules, preferably for the study of malfunctions or pathologies affecting cerebellar granules.

Another object of the present invention is a culture system or method for in vitro culture for use in gene and cell therapy approaches to cerebellar diseases caused by damage or neurodegeneration.

All the objects of the invention are better defined in the claims, in which the dependent claims describe preferred embodiments of the invention and form an integral part of this description.

### BRIEF DESCRIPTION OF THE FIGURES

FIG. 1 diagrammatically illustrates the method for in vitro culture of GCP cells which is the object of this invention.

FIG. 2 shows the average spheres obtained by culturing cells disaggregated from the cerebellum in the presence of several factors. The graph shows how the condition that is the object of the present invention has a high clonogenic power after 7 days of culture.

In the figures, NS refers to neurospheres.

FIG. 3 shows phase contrast microscope images representative of different neurosphere cultures.

FIG. 4 shows the expression of molecular biomarkers of the Shh pathway, Gli1 and N-Myc, monitored by Western Blot analysis under different culture conditions. The expression of β-actin normalises loading. The image shows how the pathway is active only in neurospheres grown in the presence of SAG or EGF+SAG, the latter condition being the object of the present invention.

FIG. 5 shows expression of the Shh pathway monitored through analysis of its expression targets through real time Q-PCR. The values are expressed as ΔCT. Consistently with FIG. 4, here again it is apparent that neurospheres grown in the presence of EGF+SAG have Shh pathway activation levels that are comparable to those of cultures grown with SAG and are even higher than the standard culture of aggregate-grown granule precursors (SGCPs).

FIG. 6 shows how the culture system according to the present invention reduces the expression of the most common differentiated granule markers, in comparison with SGCPs culture (Pax6, Cntn2, Zic1, NeuroD1, Zic3,). Conversely it maintains a high expression of granule precursor markers (Atoh1). The gene expression values, obtained through real time Q-PCR, have been expressed as ΔCT.

FIG. 7 shows images of the neuronal phenotype and expression of the β3-tubulin neuronal marker, acquired by contrast and fluorescence microscopy, in the neurospheres grown under the appropriately differentiated conditions indicated (1% foetal serum or 1% foetal serum+Vitamin A) respectively. The neurospheres of the present invention are able to differentiate by acquiring a clearly neuronal phenotype, and are positive for β3-tubulin expression in a similar way to neurospheres grown in the presence of SAG and differentiated under the same conditions.

FIG. 8 shows the average for spheres obtained from cultures grown in the presence of SAG or EGF+SAG over time. As can be seen from the graph, the EGF+SAG condition which is the object of the present invention maintains a high clonogenic power several weeks after being grown, while the condition with only SAG undergoes progressive decline.

FIG. 9 shows representative photos of cultures grown in the presence of SAG or EGF+SAG (as in FIG. 8) 4 weeks after culture.

FIG. 10 shows maintenance of the Shh pathway activity as evidenced by the expression of molecular biomarkers such as Gli1 and N-Myc in neurospheres grown in the presence of EGF+SAG for 6 weeks.

FIG. 11 shows a higher expression of Nestin and SOX2 stem markers in neurospheres grown in the presence of EGF+SAG than in cultures grown in the presence of SAG alone. The values, obtained through real time Q-PCR, have been expressed as a ratio with respect to those of cultures grown in the presence of SAG only.

### DETAILED DESCRIPTION OF THE INVENTION

A first object of the present invention is an in vitro culture system for GCP cells according to claim 1.

According to the present invention the term GCP cells (granule cell progenitors) means cerebellar granule cell precursors.

The term ‘culture medium’ according to this invention means a preferably solid medium, such as a polystyrene flask suitable for cell cultures.

By mammalian GCP cells, according to this invention, are meant GCP cells derived from the cerebellum of normal mammalian animals (including humans) or transgenic mammalian animals (excluding humans). Preferably the GCP cells of the system and method according to this invention are the GCP cells of rodents, preferably non-transgenic mouse cells preferably obtained from brains taken on the seventh day of postnatal life (P7). The system and method according to the present invention preferably use the following mouse strains: CD1, C57Bl/6 and 129.

The GCP cells according to the system and method according to this invention are not GCP cells derived from human embryos.

The GCP cells according to this invention are typically grown without substrate adherence and typically as neurospheres (also referred to as NS).

The culture medium according to this invention includes at least SAG (Smoothened Agonist) and EGF (Epidermal Growth Factor).

In the culture medium according to this invention SAG is typically in a concentration range between 100 nM and 1 μM, preferably in a concentration of 200 nM, whereas EGF is typically in a concentration range between 200 nM and 1 μM, preferably in a concentration of 1 μM. The medium according to this invention may contain components other than SAG and EGF and in particular at least one additional component chosen from DMEM-F12 (i.e. Dulbecco's Modified Eagle Medium/Nutrient Mixture F-12), glucose, Vitamin A-free B27, insulin, NALC (i.e. N-acetyl-L-cysteine), heparin, and at least one antibiotic.

According to this invention an antibiotic typically means penicillin and/or streptomycin. In a preferred aspect of the invention the medium used here contains SAG, EGF, DMEM-F12 (i.e. Dulbecco's Modified Eagle Medium/Nutrient Mixture F-12), glucose, Vitamin A-free B27 supplement, insulin, NALC (i.e. N-acetyl-L-cysteine), heparin, penicillin, and streptomycin.

In a preferred aspect of the present invention DMEM-F12 is the main component of the culture medium used here; to this are added glucose in a concentration of typically 3 mg/ml; Vitamin A-free B27 supplement used at a final concentration of typically 1× (in comparison with the concentration 50×; in fact the B27 purchased from Thermofischer, code 12587010, is then diluted 50 times in the final solution); insulin in a concentration of typically 50 μg/ml; NALC in a concentration of typically 60 g/ml; heparin in a concentration of typically 2 μg/ml; as regards antibiotics, penicillin in a concentration of typically 100 units/ml and streptomycin in a concentration of typically 0.1 mg/ml.

Mammalian GCP cells in the in vitro culture system of this invention can be expanded, driven to proliferate or grown and/or propagated and are typically in spheroidal form (neurospheres, NS) and, according to the in vitro culture method described below, are maintained in a state of unlimited proliferation and self-regeneration, measurable by clonogenicity and continuous dilution assays, while maintaining a gene/biochemical expression profile similar to fresh GCP cell cultures. As noted above, GCP cells obtained in vitro according to this system and method (described below) might be re-integrated into the brain with high tolerance and can be easily frozen and thawed without showing signs of degradation, which promotes their marketability.

The mammalian GCP cells in the system and method according to the present invention form neurospheres whose morphology or degree of confluence can be monitored by transmission and/or scanning light microscopy.

The in vitro culture system according to this invention also allows specific mammalian GCP cell markers such as Atho1, Gli1, N-Myc to be assayed qualitatively and quantitatively by Western Blot, quantitative RT-PCR and immunofluorescence.

The method for in vitro culture of GCP cells is the subject of claim 5.

In said method ‘GCP cells’ means mammalian GCP cells as defined above; the same applies to the terms ‘culture support and ‘culture medium’, which have the same meaning as above for the in vitro culture system.

Step (a) in isolating GCP cells from the mammalian cerebellum in the in vitro culture method according to the present invention may comprise shredding the mammalian cerebellum in a buffer system that may contain Hank's balanced saline solution (HBSS), glucose, DNase and at least one antibiotic.

By “antibiotic” is preferably meant one of the antibiotics mentioned above (penicillin and/or streptomycin).

Preferably, in the HBSS buffer system, glucose is typically added at a final concentration of 5 mg/ml, penicillin typically in a concentration of 100 units/ml and streptomycin typically in a concentration of 0.1 mg/ml. In the above solution, after coarse trituration using serological pipettes the brains are centrifuged at 1,300 rpm (˜190×g) for 5 minutes. The pellets thus obtained are resuspended in the same buffer system (HBSS, glucose and antibiotics) with the addition of DNase in a concentration of 1.3 U/ml, where they remain for 30 minutes at room temperature with passage through a Pasteur pipette to promote disaggregation for at least 5 minutes. After a second centrifuging at 1,300 rpm (˜190×g) for 5 minutes the GCP cells thus obtained can be resuspended in their final medium and then counted.

Subsequently, according to step b) of the in vitro culture method according to the present invention, the GCP cells obtained in step a) are placed on a culture medium or plated and are in a culture medium at a dilution typically in the range of 10,000 to 30,000 cells/cm², preferably 16,000 cells/cm².

The culture medium in the in vitro method according to the present invention is preferably DMEM-F12, Glucose, Vitamin A-free B27, insulin, NALC, heparin, at least one antibiotic, SAG and EGF.

Step c) of the in vitro culture method according to the present invention, enrichment of GCP cells and/or propagation of GCP cells by dilution, can typically be performed at a temperature of 37° C. and in the presence of 5% CO₂. The GCP spheroidal cells can be enriched and/or propagated for an unlimited period. The GCP cells in step c) are also typically dissociated periodically with a solution containing proteolytic and collagenolytic enzymes (commercially known as Accutase®), typically at least twice a week. Following treatment with Accutase® the cells can typically be centrifuged, re-suspended in their maintenance/culture medium and diluted with a 1:2 or 1:3 dilution if appropriate.

One object of the present invention is use of the culture system or method for in vitro culture as described above to generate in vitro cellular models for study of the pathophysiology of cerebellar granules, preferably for the study of malfunctions or pathologies affecting cerebellar granules.

According to this object, the in vitro culture system or method may be useful for providing/generating a reproducible and representative cell model with which to investigate mechanisms leading to the development, degeneration and transformation of GCP, in a context as close as possible to physiology but autonomous from the point of view of the cell (cell autonomous).

After differentiation, the cell model may also be a useful tool for studying the pathophysiological characteristics of cerebellar granules (formation of synapses, neurotransmission, etc.).

The in vitro culture method according to this invention may also be applied to the production of GCP cell lines including those from murine models of degenerative diseases of the cerebellum, providing a valuable aid to research on these diseases.

The in vitro culture system or method according to the present invention is also useful for providing primary cellular control models for comparing the toxicity of old and new drugs on neoplastic granules in comparison with normal proliferating or differentiated and non-proliferating granules, allowing the undesirable effects of in vitro treatments to be estimated prior to in vivo studies.

In a particularly preferred aspect, the in vitro culture system or method according to this invention may be used to generate in vitro models of malfunctions or diseases affecting cerebellar granules.

In another particularly preferred aspect, the in vitro culture system or method according to this invention may also be used in a method for screening drugs or substances that may act on cerebellar granules.

One object of the present invention is a system or method for in vitro culture as described above for use in gene therapy and cell therapy approaches to brain diseases caused by damage or neurodegeneration.

In this respect, cerebellar disease caused by damage or neurodegeneration typically relates to Ataxia Telangiectasia, Nijmegen syndrome, Joubert syndrome, Dandy-Walker syndrome, spinocerebellar ataxia, or other diseases of the cerebellum.

The system and method of culture according to this invention makes it possible to prepare cultures of GCP cells which, after enrichment, may be re-implanted into the cerebellum of an appropriate developing or adult mammal. Such GCP cells find genetic engineering applications, after in vitro correction by gene transfer or gene editing of the genetic defect of GCP from diseased individuals, in in vitro enrichment according to the in vitro culture method according to this invention and subsequent re-implantation in the cerebellum.

The system and the in vitro method described above will be described in more detail with reference to the non-limiting examples described below.

### EXAMPLE 1

### Method for In Vitro Culture According to the Present Invention

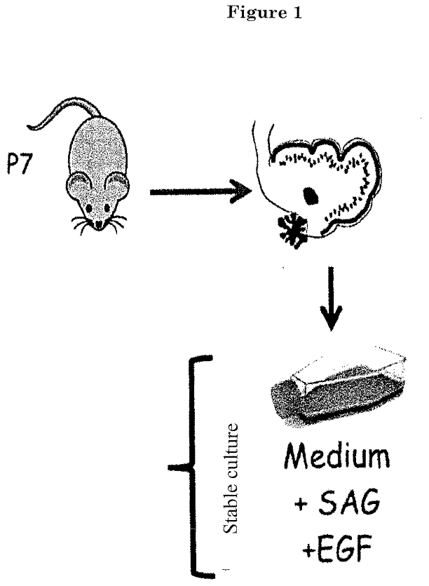

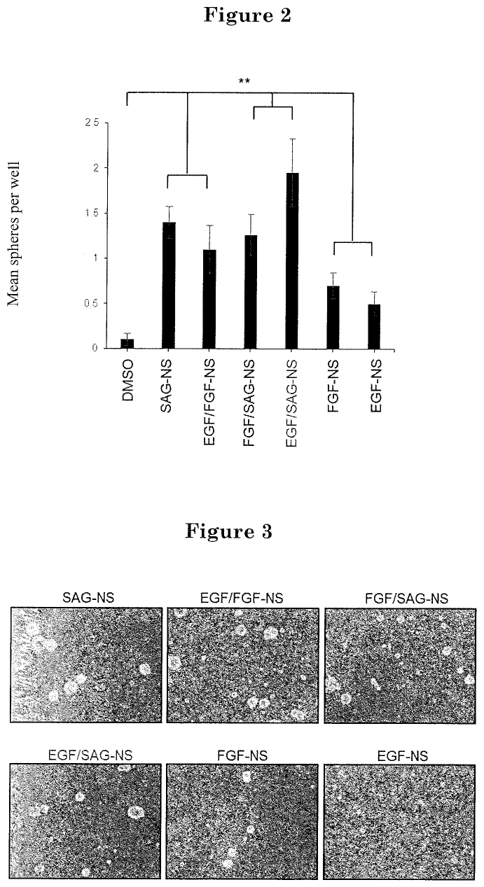

After surgical resection from a mouse on the seventh day of postnatal life (P7) the cerebellum was mechanically shredded in a buffer containing HBSS, glucose and antibiotics, and DNase (at concentrations as defined above) and the individual cells thus obtained were counted and plated at a dilution of 16000 cells/cm² on a medium composed of DMEM-F12, glucose, Vitamin A-free B27, insulin, NALC, heparin, antibiotics (at concentrations as defined above) and SAG (200 nM) and EGF (1 μM). On this medium the cells can be enriched and maintained for an unlimited time, after periodical dissociation of the spheres with Accutase® (2-3 times per week) and propagation by dilution (to prevent excessive growth and consequent death from nutrient deficiency and hypoxia). The reaction conditions were as described above. The in vitro culture method according to the present invention is shown in FIG. 1.

### Results

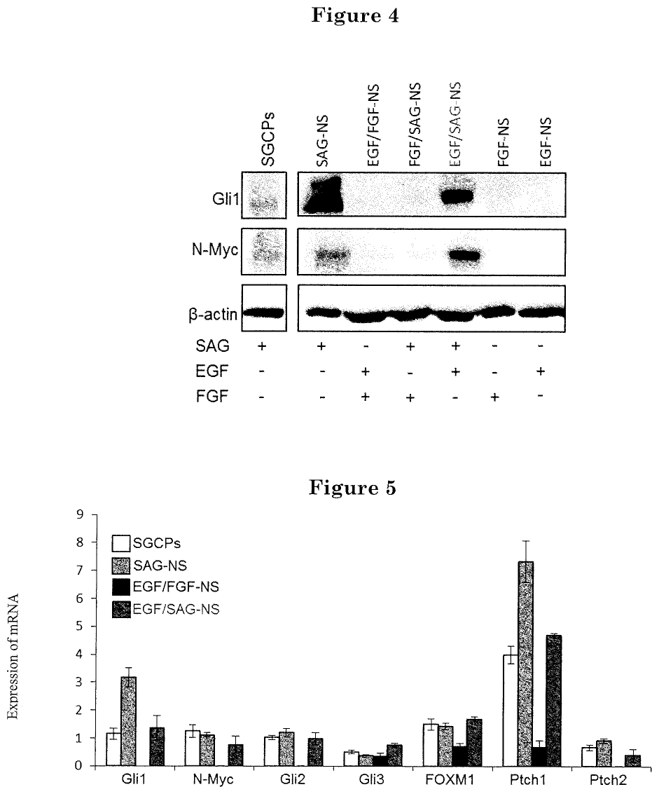

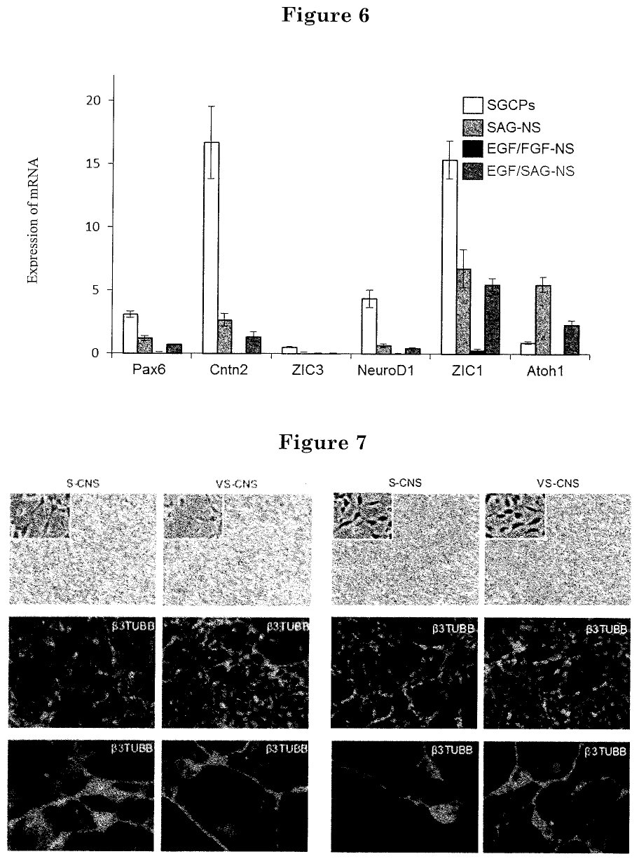

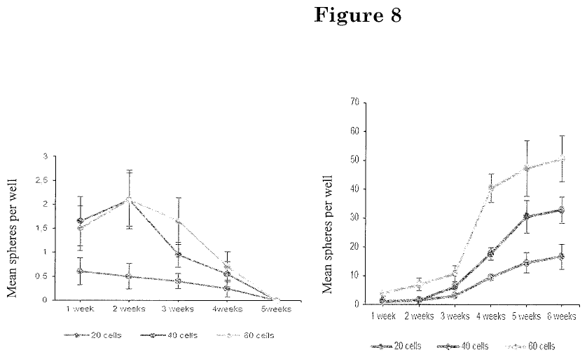

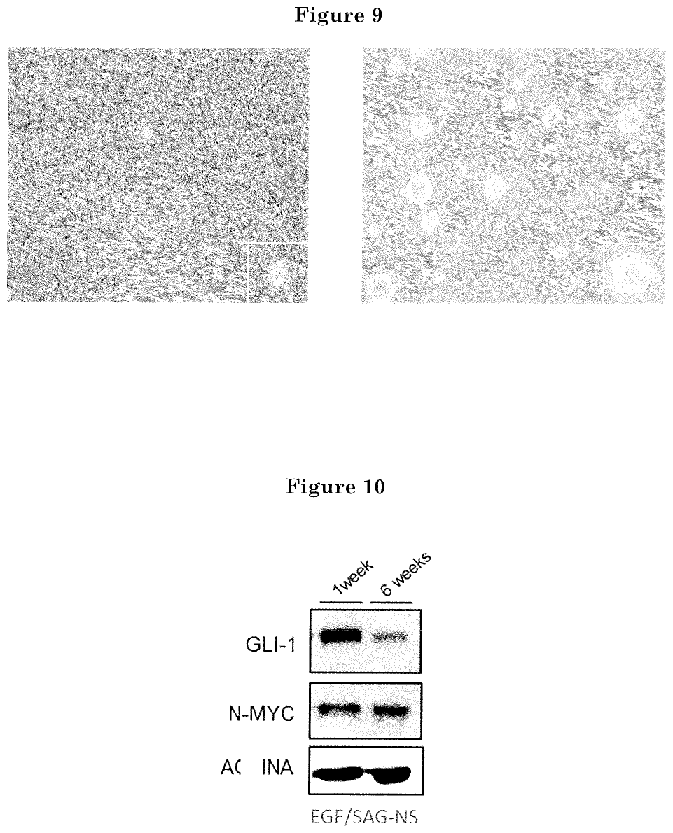

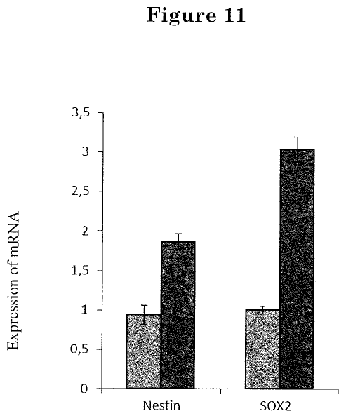

It has been observed that in GCP cells SAG maintains a high expression of the Shh pathway (monitored by the expression of Gli1 and N-MYC proteins at different times after culture) while EGF ensures its unlimited proliferative power. In addition, analysis of stem marker expression showed that the administration of EGF in addition to SAG advantageously induces higher levels of SOX2 transcription factor and Nestin protein in GCP cells than those observed in transient cultures of agglomerated or suspended GCP cells with SAG, with a significant impact on the ability of GCP cells to remain stable over time. It should be noted that our results are closely related to the synergistic effect of SAG and EGF in determining and maintaining GCP cells. In fact, the presence of EGF alone leads to the isolation of another type of cell population, which does not express the markers typical of GCP (FIG. 4). On the other hand, the presence of SAG alone does not ensure long-term survival of the culture, as is also highlighted in FIG. 8. Differentiation experiments conducted on stabilised cell lines using the in vitro culture method according to the present invention have shown that the vast majority of cells are positive for expression of the specific neuronal marker beta-3-tubulin, demonstrating that our culture is strongly enriched with a neuronal and not a glial component (FIG. 7).

1. An in vitro culture system for cerebellar granule cell precursors (GCP) comprising:
a culture support,
a plurality of mammalian GCP cells, and
contained in or on the culture support, a culture medium comprising at least SAG (Smoothened agonist) and EGF (Epidermal Growth Factor).

2. The in vitro culture system according to claim 1 in which mammalian GCP cells comprise GCP cells from rodents.

3. The in vitro culture system according to claim 1, further comprising at least one component selected from the group consisting of: DMEM-F12, Dulbecco's Modified Eagle Medium/Nutrient Mixture F-12, glucose, Vitamin A-free B27 supplement, insulin, N-acetyl-L-cysteine heparin and at least one antibiotic.

4. The in culture system of claim 1, wherein the culture medium comprises SAG, EGF, Dulbecco's Modified Eagle Medium/Nutrient Mixture F-12, glucose, Vitamin A-free B27 supplement, insulin, N-acetyl-L-cysteine, heparin, penicillin and streptomycin.

5. A method for the in vitro culture of cerebellar granule cell precursor (GCP) cells comprising:
(a) isolating of GCP cells from a mammalian cerebellum,
(b) placing the GCP cells obtained in step (a) on or in a culture medium comprising at least SAG and EGF, and
(c) enriching and/or propagating the GCP cells by dilution.

6. The method of claim 5, wherein step a) comprises shredding the mammalian cerebellum.

7. The method of claim 5, wherein the GCP cells in the culture medium in step b) are at a dilution in the range of between about 10,000 to 30,000 cells/cm2.

8. The method of claim 5, wherein the GCP cells in step c) are periodically dissociated with a solution comprising proteolytic and collagenolytic enzymes.

9. The method according to claim 5, further comprising generating in vitro models for studying the pathophysiology of cerebellar granules, or studying a malfunction or a disease affecting cerebellar granules.

10. The method according to claim 5, further comprising re-integrating the enriched or propagated GCP cells into a brain and studying a degenerative disease of a cerebellum in a mouse model.

11. The method according to claim 5, further comprising using the enriched or propagated GCP cells to provide a primary cellular model of a cerebellar tumor and controls for comparing the toxicity of old and new drugs on neoplastic granules in comparison with proliferating or differentiated and non-proliferating normal granules.

12. The method according to claim 5, further comprising using the enriched or propagated GCP cells for screening for drugs or substances that may act on a cerebellar granule.

13. The method according to claim 5, further comprising using the enriched or propagated GCP cells for studying in gene therapy or cell therapy approaches for a brain disease caused by damage or neurodegeneration.

14. The in vitro culture system of claim 2, wherein the rodent is a mouse.

15. The in vitro culture system of claim 14, wherein the mouse is a non-transgenic mouse.

16. The in vitro culture system of claim 1, wherein the plurality of mammalian GCP cells is obtained from brains taken on the seventh day of postnatal life.

17. The method of claim 6, wherein the mammalian cerebellum is shredded in a buffer system comprising Hank's balanced saline solution (HBSS), glucose, DNase and at least one antibiotic.

18. The method of claim 7, wherein the GCP cells in the culture medium in step b) are at a dilution of about 16,000 cells/cm2.

19. The method of claim 13, wherein the brain disease is a disease of the cerebellum.

20. The method of claim 13, wherein the brain disease is selected from the group consisting of: Ataxia Telangiectasia, Nijmegen syndrome, Joubert syndrome, Dandy-Walker syndrome, spinocerebellar ataxia.
